(19) United States
(12) Patent Application Publication
(10) Pub. No.: US 2025/0391571 A1
(43) Pub. Date: Dec. 25, 2025

(54) SYSTEM AND METHOD FOR PROVIDING A TRUE HEALTH-WELLNESS STATUS OF A USER

(71) Applicant: Girish KRISHNAMURTHY, Bengaluru (IN)

(72) Inventors: Girish KRISHNAMURTHY, Bengaluru (IN); Srikrishna SESHADRI, Bangalore (IN); Dr Nayeem AHMED, Bangalore (IN)

(21) Appl. No.: 18/878,829

(22) PCT Filed: Jun. 23, 2023
(86) PCT No.: PCT/IN2023/050607

§ 371 Date: Dec. 24, 2024

(30) Foreign Application Priority Data
Jun. 24, 2022 (IN) 202241036370

(51) Int. Cl. G16H 50/30; G16H 10/60; G16H 50/20; G16H 70/20; G16H 80/00

(52) U.S. Cl. CPC G16H 50/30; G16H 10/60; G16H 50/20; G16H 70/20; G16H 80/00

(57) ABSTRACT

A system and method for providing a true health-wellness status of a user, where such true health-wellness status is a holistic and/or a specific true health-wellness status. The method encompasses: receiving, by a transceiver unit [102] from a storage unit [108], a user data; analysing, by the processing unit [104], the received data, based at least on a first set of pre-set rules to generate a corresponding composite score for one or more health based dimensions of the user; automatically generating, by the processing unit [104], a health-wellness score of the user based on a weighting of the corresponding composite score based on a second set of pre-set rules; providing, by the processing unit [104], to at least one of the user and a healthcare provider, the true health-wellness status of the user based on the health-wellness score of the user.

### TECHNICAL FIELD

The present invention generally relates to digital healthcare technologies and more particularly to systems and methods for providing a true health-wellness status of a user.

### BACKGROUND OF THE DISCLOSURE

The following description of the related art is intended to provide background information pertaining to the field of the disclosure. This section may include certain aspects of the art that may be related to various features of the present disclosure. However, it should be appreciated that this section is used only to enhance the understanding of the reader with respect to the present disclosure, and not as admissions of the prior art.

Over the past few years digital healthcare technologies have been enhanced to a great extent and currently these technologies are supporting patients in checking the status of a disease or chronic condition. The digital healthcare technologies are important, as while helping patients in monitoring and managing diseases and chronic conditions, these technologies also prevent various diseases and lower healthcare costs for the patients. Also, it is important to note the WHO's definition of health i.e., “a state of complete physical, mental and social well-being, and not merely the absence of disease” has been widely used and established. Yet, only a few people have attempted to operationalize this term so that it may be used to assess people's health. Before an individual's or a population's health level or health status can be assessed, an operational definition of health is required. As the focus of medical and health care has switched from decreasing mortality and increasing longevity to improving health-related quality of life, defining such a term has grown more complex. When it comes to acute conditions that are potentially fatal, length of life is the ultimate measure of health. However, in chronic conditions such as including but not limited to diabetes where palliative medicines or therapies that may prevent further deterioration are used, the meaningful measure of health status is likely to include health domains other than duration of life.

Wellness, on the other hand, is defined by The Global Wellness Institute as “the active pursuit of activities, choices and lifestyles that lead to a state of holistic health”. There are two important aspects to this definition. First, wellness is not a passive or static state but rather an “active pursuit” that is associated with intentions, choices and actions as we work toward an optimal state of health and wellbeing. Second, wellness is linked to holistic health—that is, it extends beyond physical health and incorporates many different dimensions that should work in harmony. Despite a much clearer definition, a solution to quantify wellness considering all its aspects is currently lacking. In the absence of a clear operational definition, several measures are being used currently that act as proxy to wellness/health or measure them partially. For instance, the health status may be currently measured based on the following:
1. Health status can be measured using clinical measures that require a physician or other trained professionals to observe and measure. Some examples are Signs (for e.g., blood sugar, blood pressure, temperature, oxygen saturation, heart sounds etc.), Symptoms (disease specific checklists) and Co-morbidity (Charlson Index (CCI score), ICED-index of co-existing disease, Clavien-Dindo Classification of Surgical Complications etc.).
2. Beyond clinical measures, another very commonly used measure is the ‘quality of life’. It measures to what extent the diseases/condition affects the routine life of an individual. Health related quality of life (HRQoL) generally measured by asking the patient directly or through various instruments. Also, the health-related quality of life measures can be used for determining a variety of diseases, medical treatments, and demographic/cultural groupings, or these can be limited to certain diseases, interventions, or population groups. HRQoL measurements are useful because they can determine the breadth of issues that patients face, identify any ongoing issues that could otherwise go unnoticed, and predict therapy success. HRQoL metrics can be paired with time spent in a certain health condition to create Quality Adjusted Life Years (QALYs). Some tools to measure HRQoL are Short form (SF)-36, SF-12, WHOQOL, EuroQoL, Nottingham health profile, Sickness Impact Profile etc. The SF-36 instrument, which consists of a 36-item self-administered questionnaire, is a commonly used tool. It is presently accepted as a gold standard measure since it gives scores on eight health categories plus two summary scores. SF-12 is a shortened version of SF-36. It is also a self-reported outcome measure assessing the impact of health on an individual's everyday life.

Currently, the health management solutions do not have a way to measure and represent the holistic health status of individuals in a manner understandable to them and also these known solutions fail to simultaneously provide actionable solutions. Additionally, as a health score of an individual that is aggregated across all dimensions has an advantage of providing a single health value for the individual, a multidimensional health assessment is required to generate such single aggregated health score. But existing health assessment solutions do not have a true multidimensional approach while measuring health and deal with one or a few components of health (such as physical health or mental health etc.) alone. Also, the existing health measurement solutions have at least the following limitations:
1. Capturing Nuances: A single health-score as provided by the existing solutions may not always reflect all the individual's health's nuances. For example, two people may have the same health-score, but one may have current acute health problems, while the other has more long-term risks.
2. Device dependence: Currently, most health scores are based on device generated data alone. Individual's subjective experiences or perceptions are not taken into account. The device dependence also increases the cost of the entire exercise.
3. Absence of patient centricity: Current health scores lack patient centricity i.e. they are built with the objective of either helping healthcare provider in making a clinical decision or for helping insurance providers in deciding premiums.
4. Lack of Uniform Standards: Data may not always mean the same thing to different people; normal ranges, cut-offs, and readings for a population vary based on different demographic factors, resulting in a lack of uniformity in the systems of the current solutions.
5. Data Interpretation: The current health management solutions do not have a way to measure and represent the holistic health status of individuals in a manner understandable to them.
6. Complexity of health data: Health data isn't only about individual elements; it can also be about an interplay of various parameters that together create meaningful insights. The currently known solutions fail to efficiently deal with the complexity of health data.

Furthermore, some of the chronic diseases (say for e.g., diabetes mellitus etc.) are one of the world's largest public health problems today. These chronic diseases have high and growing prevalence and cause diverse and extensive morbidity, impacting not just individuals, but health systems and national economies. Therefore, constantly measuring and managing one's health status is of utmost importance. Further, in view of at least the above-mentioned limitations of the existing digital healthcare solutions, presently these existing solutions fail to efficiently and effectively capture and provide a holistic true health-wellness status at an individual level. Therefore, to overcome the limitations of the current solutions, there is a need in the art to provide a method and system for providing a true health-wellness status of a user.

### SUMMARY OF THE DISCLOSURE

This section is provided to introduce certain objects and aspects of the present invention in a simplified form that are further described below in the detailed description. This summary is not intended to identify the key features or the scope of the claimed subject matter.

Thus, a first object of the present disclosure is to provide a method and a system for providing a true health-wellness status of a user that overcomes the limitations of the existing approaches. Another object of the present disclosure is to provide a health-wellness information at an individual level that is both dynamic and holistic about the individual's health. Yet another object of the present disclosure is to enable individuals to make decisions regarding their health. Yet another object of the present disclosure is to motivate people to reach their optimal health status. Yet another object of the present disclosure is to provide support in clinical decision making. Yet another object of the present disclosure is to ensure an on-time health-wellness care delivery. Yet another object of the present disclosure is to improve overall healthcare experience for the users. Yet another object of the present disclosure is to cater different user personas by presenting information about individual's health to both individuals and healthcare providers.

In order to achieve at least one of the objectives as mentioned above, one aspect of the present invention relates to a method for providing a true health-wellness status of a user of an electronic device. The method comprises receiving, by a transceiver unit from a storage unit, a user data comprising at least one of a demographics data, a physical health data, a mental and emotional health data, and a behavioural health data. Further the method comprises analysing, by the processing unit, the user data comprising at least one of the demographics data, the physical health data, the mental and emotional health data, and the behavioural health data based at least on a first set of pre-set rules. The method further comprises generating, by the processing unit, a corresponding composite score for one or more health based dimensions of the user, based on the analysis of the user data. Further, the method comprises automatically generating, by the processing unit, a health-wellness score of the user based at least on the corresponding composite score for one or more health based dimensions. The method further comprises automatically generating, by the processing unit, the true health-wellness status of the user based on the health-wellness score of the user. Finally, the method comprises providing, by the transceiver unit, to at least one of the electronic device, and a healthcare provider device, the true health-wellness status of the user.

Another aspect of the present invention relates to a system for providing a true health-wellness status of a user of an electronic device. The system comprises at least a transceiver unit, a storage unit, and a processing unit connected to the transceiver unit. The transceiver unit is configured to receive, from the storage unit, a user data comprising at least one of a demographics data, a physical health data, a mental and emotional health data, and a behavioural health data. Further, the processing unit is configured to analyse the user data comprising at least one of the demographics data, the physical health data, the mental and emotional health data, and the behavioural health data based at least on a first set of pre-set rules. The processing unit is further configured to generate a corresponding composite score for one or more health based dimensions of the user, based on the analysis of the user data. Further, the processing unit is configured to automatically generate a health-wellness score of the user based at least on the corresponding composite score for one or more health based dimensions. Further, the processing unit is configured to automatically generate the true health-wellness status of the user based on the health-wellness score of the user. Further, the processing unit is configured to provide, via the transceiver unit, to at least one of the electronic device, and a healthcare provider device, the true health-wellness status of the user.

### BRIEF DESCRIPTION OF DRAWINGS

The accompanying drawings, which are incorporated herein, and constitute a part of this disclosure, illustrate exemplary embodiments of the disclosed methods and systems in which like reference numerals refer to the same parts throughout the different drawings. Components in the drawings are not necessarily to scale, emphasis instead being placed upon clearly illustrating the principles of the present disclosure. Some drawings may indicate the components using block diagrams and may not represent the internal circuitry of each component. It will be appreciated by those skilled in the art that disclosure of such drawings includes disclosure of electrical components, electronic components or circuitry commonly used to implement such components.

FIG. 1 illustrates an exemplary block diagram of a system [100] for providing a true health-wellness status of a user, in accordance with exemplary embodiments of the present invention.

FIG. 2 illustrates an exemplary method flow diagram [200], for providing a true health-wellness status of a user, in accordance with exemplary embodiments of the present invention.

FIG. 3 illustrates an exemplary representation providing an exemplary true health-wellness status, in accordance with exemplary embodiments of the present invention.

FIG. 4 illustrates an exemplary input output map of a system [100] for providing a true health-wellness status of a user, in accordance with exemplary embodiments of the present invention.

The foregoing shall be more apparent from the following brief description of the exemplary embodiments of the disclosure.

### DETAILED DESCRIPTION OF THE DISCLOSURE

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address any of the problems discussed above or might address only some of the problems discussed above.

The ensuing description provides exemplary embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the disclosure as set forth.

Specific details are given in the following description to provide a thorough understanding of the embodiments. However, it will be understood by one of ordinary skill in the art that the embodiments may be practiced without these specific details. For example, circuits, systems, processes, and other components may be shown as components in block diagram form in order not to obscure the embodiments in unnecessary detail.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

As used herein, a “processing unit” or “processor” or “operating processor” includes one or more processors, wherein processor refers to any logic circuitry for processing instructions. A processor may be a general-purpose processor, a special purpose processor, a conventional processor, a digital signal processor, a plurality of microprocessors, one or more microprocessors in association with a DSP core, a controller, a microcontroller, Application Specific Integrated Circuits, Field Programmable Gate Array circuits, any other type of integrated circuits, etc. The processor may perform signal coding data processing, input/output processing, and/or any other functionality that enables the working of the system according to the present disclosure. More specifically, the processor or processing unit is a hardware processor.

As used herein, “a user equipment”, “a user device”, “a smart-user-device”, “a smart-device”, “an electronic device”, “a mobile device”, “a handheld device”, “a wireless communication device”, “a mobile communication device”, “a communication device” may be any electrical, electronic and/or computing device or equipment, capable of implementing the features of the present disclosure. The user equipment/device may include, but is not limited to, a mobile phone, smart phone, laptop, a general-purpose computer, desktop, personal digital assistant, tablet computer, wearable device or any other computing device which is capable of implementing the features of the present disclosure. Also, the user device may contain at least one input means configured to receive an input from a transceiver unit, a processing unit, a storage unit and any other such unit(s) which are required to implement the features of the present disclosure.

As used herein, “storage unit” or “memory unit” refers to a machine or computer-readable medium including any mechanism for storing information in a form readable by a computer or similar machine. For example, a computer-readable medium includes read-only memory (“ROM”), random access memory (“RAM”), magnetic disk storage media, optical storage media, flash memory devices or other types of machine-accessible storage media. The storage unit stores at least the data that may be required by one or more units of the system to perform their respective functions.

As used herein, a “user interface” typically includes an output device in the form of a display, such as a liquid crystal display (LCD), cathode ray tube (CRT) monitors, light emitting diode (LED) screens, etc. and/or one or more input devices such as touchpads or touchscreens. The display may be a part of a portable electronic device such as smartphones, tablets, mobile phones, wearable devices, etc. They also include monitors or LED/LCD screens, television screens, etc. that may not be portable. The display is typically configured to provide visual information such as text and graphics. An input device is typically configured to perform operations such as issuing commands, selecting, and moving a cursor or selector in an electronic device.

As disclosed in the background section, existing technologies have many limitations and in order to overcome at least some of the limitations of the prior known solutions, the present disclosure provides a solution for providing a true health-wellness status of a user. In an instance, a true health-wellness status of a user including at least a single index of the user's health may be determined based on aggregating score of a multidimensional measure of the user's health. In an exemplary implementation of the present disclosure these aggregated scores can be derived through a combination of summated ratings and a weighting technique that generates estimates for various levels of health. Also, if the measure or index is intended for routine or periodic use it should be appropriate, relevant, acceptable, personalized and/or patient centric, have both holistic and specific components, reliable, brief and simple to administer, actionable and comparable etc. The solution as disclosed in the present disclosure, considers these parameters to provide a general and/or a disease-specific, dynamic, visual and/or numerical representation of individual's true health-wellness status. Also, the solution as disclosed in the present disclosure provides two levels of information, i.e., 1) a holistic, general true health-wellness status/health-wellness score that considers multiple dimensions of health of an individual, and 2) a specific health and wellness score/status that could be pertaining to individual dimensions of health or the disease/condition that the individual has. In a preferred implementation the present disclosure provides a solution for providing a diabetes specific true health-wellness status of a user, but the disclosure is not limited thereto, and in an implementation, a true health-wellness status specific to some other disease may also be provided based on the implementation of the features of the present invention. Further, in such preferred implementation, the diabetes specific true health-wellness status of the user may be generated by using an aggregated score of a multidimensional measure which includes glycaemic control, risk of complications, treatment adherence etc.

More specifically, in an implementation, to determine a health and wellness score/status, once a diagnosis is made of a general health and/or a disease say for e.g., diabetes, and an option (such as a suitable care service) is selected for implementation of the features of the present disclosure, then the present solution encompasses completing and storing relevant investigations and questionnaire assessments related to the diagnosed general health conditions and/or the diagnosed disease. These stored data points are then used, along with a past medical data, and these data are then structured and presented as a health and wellness status represented in three categories: Ideal, Optimal, Sub-Optimal. Also, in an implementation of the present disclosure the health and wellness score/status may indicate at least one of a Physical Activity score, Habits score, Diet score, Risk Factor Score, Mental Health Score, Acute Illness Score, Chronic Illness Score, Pain Score, Medication Score, etc., wherein these scores together aim to provide a dynamic and comprehensive view of an individual's health.

Also, the present solution comprises providing the true health-wellness status of a user to the user and/or to one or more healthcare providers. Therefore, in the present solution two distinct needs may be considered while presenting the true health-wellness status i.e., one for the individuals and one for the healthcare providers. In an instance the true health-wellness status is provided to the individuals in a manner that is easily understandable, for example it may be represented either using a visual scale or colour codes. Simultaneously, the true health-wellness status of a patient may be provided to the healthcare providers in the form of detailed scores, timelines, trends etc. for monitoring the patient and tracking the patient's progress over time on a continuous basis.

Hereinafter, exemplary embodiments of the present disclosure will be described with reference to the accompanying drawings so that those skilled in the art can easily carry out the present disclosure.

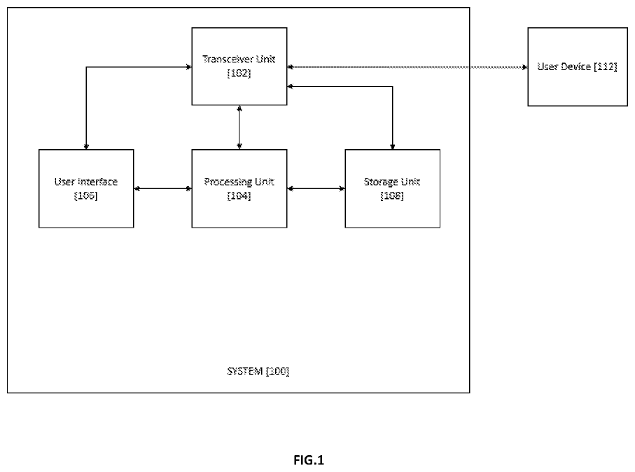

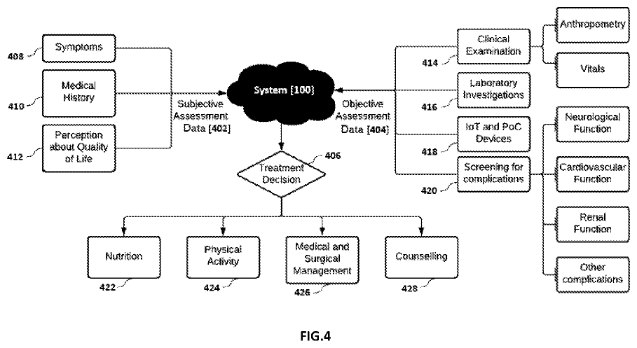

Referring to FIG. 1, an exemplary block diagram of a system [100] for providing a true health-wellness status of a user of an electronic device is shown in accordance with exemplary embodiments of the present invention. The system [100] comprises at least one transceiver unit [102], at least one processing unit [104], at least one user interface [106] and at least one storage unit [108]. Also, all of the components/units of the system [100] are assumed to be connected to each other unless otherwise indicated below. Also, in FIG. 1 only a few units are shown, however, the system [100] may comprise multiple such units or the system [100] may comprise any such numbers of said units, as required to implement the features of the present disclosure. Further, in an implementation, the system [100] may be present in a server device to implement the features of the present invention. Also, FIG. 4 depicts an exemplary input output map of a system [100] for providing a true health-wellness status of a user, in accordance with exemplary embodiments of the present invention. For clear explanation, FIG. 1 and FIG. 4 may be explained in conjunction with each other wherever necessary.

The system [100] is configured to provide the true health-wellness status of the user with the help of the interconnection between the components/units of the system [100]. The present disclosure via the true health-wellness status provides two levels of information, i.e., 1) a holistic, general true health-wellness status/health-wellness score that considers multiple dimensions of health of an individual, and 2) a specific health and wellness score/status that could be pertaining to individual dimensions of health or the disease/condition that the individual has.

The transceiver unit [102] of the system [100] is configured to receive, from the storage unit [106], a user data comprising at least one of a demographics data, a physical health data, a mental and emotional health data, and a behavioural health data. In an implementation, the demographics data, the physical health data, the mental and emotional health data, and the behavioural health data further comprises one or more parameters associated with the user. In an implementation, the user data is provided by the user via the electronic device. In an implementation, the user data is provided by a healthcare provider via the electronic device and/or via any other smart device connected with the electronic device. In an implementation, the storage unit [106] may be integrated in the system [100] or may be present separately and operably coupled to the system [100]. The storage unit [106] may be connected to the electronic device. In an implementation, the electronic device may be a user device of the user, a user device of a healthcare provider, or a remote storage device configured to store data received from one or more user devices, wherein the user device may be any device that is capable of implementing the features of the present invention. In an implementation, the transceiver unit [102] receives the data at a wellness configurator unit. The wellness configurator unit, in an implementation, may be a unit similar to the processing unit [104] and/or may be implemented in the processing unit [104]. Further, the demographics data associated with the user includes, but not limited to, a data related to the user that is determined based on a study of a population based on factors such as age, race, and gender etc. Also, the demographic data comprises at least a socio-economic status of the user, where in an example, the socioeconomic status/information may include a data determined based on an employment, education, income, marriage rates, birth and death rates, and more factors.

Also, in an implementation, the one or more parameters are configurable based on one of an automatic input and a manual input. For example, the user of the system [100] may select the parameter(s) to focus on while assessing the health of the assessee (i.e., the one whose health is assessed). Also, the user of the system [100] may be the same person whose health is assessed or may be a healthcare provider who is providing services to the user by using the system [100]. In an example, the user may select one or more parameters to focus on, such as Hemoglobin (Hb) level of the blood of the assessee, blood pressure of the assessee, oxygen level of the assessee, etc. This may be done via manual input of the user. Further, in another implementation, the user may select a disease to assess in the assessee. By simply selecting an option of that disease, the system [100] automatically selects corresponding one or more parameters to focus on. Some exemplary care indicators may include, but not limited to, demographics, anthropometry, socio-economic status, physical activity, diet, sleep, mental health, blood pressure, fasting sugar level, glycaemic profile, kidney function, lipid profile, macrovascular complications, microvascular complications, substance abuse, pain scale, symptom checker, medication checker, treatment adherence, etc.

The transceiver unit [102] of the system [100] is also configured to receive from the storage unit [106], a physical health data associated with the user, wherein the physical health data comprises at least an anthropometry data, a general physical examination data and a systemic examination data etc. Also, the transceiver unit [102] of the system [100] is configured to receive from the storage unit [106], a mental and emotional health data and a behavioural health data associated with the user. The behavioural health data comprises at least a substance abuse data, and a treatment adherence data. Also, in an implementation, the user data is based on a primary diagnosis and other comorbidities of the user to make the assessment patient/user centric and customized for each user's/individual's health need. Further, the data received by the transceiver unit [102] from the storage unit [106] is used to determine a health-wellness score (for instance, a general or a disease specific health-wellness score) of the user, and in an implementation, the storage unit [106] may receive such data (i.e., the data to be transmitted to the transceiver unit [102]) from various sources including, but not limited to, one or more medical history forms provided by the user and/or the healthcare provider, one or more IoT and Point of Care devices, one or more validated assessment tools/questionnaires, one or more Lab reports, and one or more subjective inputs from Clinical Staff, one or more wearable gadgets of the user, etc. In an implementation, the storage unit [106] receives the data via the transceiver unit [102] and/or the processing unit [104]. Therefore, the data may be received at the transceiver unit [102] from various sources that may be device based sources and/or device-less sources. Also, the data received by the transceiver unit [102] from the storage unit [106] includes both subjective and objective data in order to obtain a result that is truly multidimensional. For example, as shown in FIG. 4, the subjective assessment data [402] and the objective assessment data [404]. The objective assessment data [404] may be obtained from clinical examination [414] (such as, anthropometry data, and other vitals of the user), medical records, lab investigations [416], internet of things (IoT) and Point of Care (POC) devices [418], screening for complications [420] (such as, neurological function, cardiovascular function, renal function, etc.) etc., where one or more members of a Care Team may coordinate with the user to schedule activities to obtain information from above mentioned sources. Also, as shown in FIG. 4, the subjective assessment data [402] may be obtained from various sources such as, but not limited to, symptoms [408] that may be provided to the system as input to the system [100] by the user, medical history of the user [410], and perception about quality of life [412], etc.

The transceiver unit [102] after receiving the data from the storage unit [106] provides the received user data to the processing unit [104] (say for e.g., to the Wellness Configurator Unit). The processing unit [104] of the system [100] is then configured to analyse the received user data, i.e., at least one of the demographics data, the physical health data, the mental and emotional health data and the behavioural health data based at least on a first set of pre-set rules. The first set of pre-set rules comprises selecting the one or more parameters to assess the patient based on a disease condition of the patient. Further, the first set of pre-set rules comprises assigning a weightage to each of the selected one or more parameters. These weightages may be assigned by a physician or a healthcare provider, or may be by the system based on historically assigned weightages. For example, the first set of pre-set rules may comprise that for the Disease A, the parameters ‘number of hours of physical activity’, ‘number of hours of meditation’, and ‘number of hours of sleep’ are selected parameters, and say, the ‘number of hours of physical activity’ is given 50% weightage, the ‘number of hours of meditation’ is given 30% weightage, and the ‘number of hours of sleep’ is given 20% weightage. A person skilled in the art would appreciate that the above values are for understanding purposes only and do not restrict or limit the disclosure in any possible manner. The processing unit [104] generates a corresponding composite score for one or more health based dimensions of the user, based on the analysis of the user data. The corresponding composite score for one or more health based dimensions is generated to determine the health-wellness score of the user. For the purposes of clear explanation, the term “corresponding composite score” indicates that a separate composite score is generated for each health-based dimension of the user.

Further, in an implementation, the corresponding composite score for the one or more health based dimensions is based on at least one of a corresponding score-weightage for each parameter of the one or more parameters. The analysis may be based on at least one of the one or more medical guidelines and a medical history of the user. The medical history of the user may also be based on a family medical history of the user. For example, a user selects “Diabetes” as the disease for assessment. From the general medical guidelines such as a set of international guidelines, or general medical practices guidelines in a country, etc., two standard tests are prescribed, say Test A and Test B. However, from the family medical history of the user or the medical history of the user, it is suggested that Test C should also be performed for the assessment of diabetes in the user's specific case. Thus, it may be notified to the user by, for example, generating a prompt by the system that Test C is also suggested to be taken by the user. Thus, in an implementation, the user can select an option indicating whether the user wants to take the suggested test into consideration or continue with the analysis based on standard tests only. In another implementation, the user is not given an option to select the test consideration, but the prompt only contains an information informing the user that parameters related to the Test C are also taken into consideration while generating results.

Based on the analysis, the processing unit [104] assigns a weightage to the one or more parameters present in the received data to determine the corresponding composite score for one or more health based dimensions (i.e., to further determine the health-wellness score), wherein initially such weightage may be assigned for determining the corresponding composite score for one or more health based dimensions (or the health-wellness score) at a beginning level and the weightage may be then modified periodically and/or dynamically as per the user's health progress, so that the composite score for one or more health based dimensions (or the user's health-wellness score) may be determined and/or tracked effectively. Here, the corresponding composite score for the one or more health based dimensions is updated periodically based on a periodical update in the user data. In an implementation, the user data is updated periodically at regular or irregular intervals. In an implementation, the updating of the corresponding composite score for the one or more health based dimensions and the updating of the user data are synchronized in a manner that the corresponding composite score(s) is updated as and when the user data is updated. Also, in an implementation, the processing unit [104] (i.e., a wellness assessment unit) receives the weighted values of the one or more parameters present in the user data along with respective one or more baseline values of said one or more parameters and one or more target values for of said one or more parameters. The Wellness Assessment Unit may be a unit similar to the processing unit [104] and is configured to implement the features of the present invention. Further, in the given implementation the processing unit [104] (i.e., the Wellness Assessment Unit) may be configured to provide the composite score for one or more health based dimensions (or the health-wellness score) for the user based at least on an analysis of the weighted values of the one or more parameters present in the received data with respect to the one or more baseline values of said one or more parameters and the one or more target values for of said one or more parameters.

Also, in an implementation the received data i.e., including but not limited to at least one of the demographics data, the physical health data, the mental and emotional health data and the behavioural health data are also analysed by the processing unit [104] based on a patient/user health data of one or more patients/users similar to the user. For example, if the user is diagnosed with diabetes or a particular health condition, then the demographics data, the physical health data, the mental and emotional health data and/or the behavioural health data of such user are analysed based on a patient health data of one or more patients that at least are also diagnosed with diabetes or the particular health condition, and the first set of pre-set rules. More specifically, the processing unit [104] compiles and scores the demographics data, the physical health data, the mental and emotional health data and/or the behavioural health data based at least on the analysis of the demographics data, the physical health data, the mental and emotional health data and/or the behavioural health data, wherein the analysis is based on the first set of pre-set rules and the patient health data of the one or more patients that are also diagnosed with the same disease and/or same health condition as that of the user, to generate the composite score for the one or more health based dimensions of the user. In the present disclosure the analysis of the data by the processing unit [104] is carried out on evidence-based outcomes. For instance, in an implementation the data is queried, and aggregated directly from an anonymized, central data repository (CDR) of similar patients. Thereafter, the processing unit [104] applies intelligent informatics methodologies (such as AI and data mining based methodologies) to extract and interpret information from substantial amounts of CDR and from subjective and/or objective assessments of user/patient health data which constitutes of demographics, assessments, laboratory test result values, procedures undertaken, diagnoses, treatment, medication as well as time etc. The methodologies applied by the processing unit [104] allows detailed data analysis of patient/user journey, to provide interactive representation of data, value-based outcomes for quality evaluations, better healthcare options and real time valuable information to support continuous care management.

The processing unit [104] after generating the corresponding composite score for the one or more health based dimensions of the user, is configured to automatically generate, the health-wellness score (i.e., the general and/or specific health-wellness score) of the user based on a weighting of the corresponding composite score. Further, the weighting of the corresponding composite score is based on a second set of pre-set rules. The second set of pre-set rules comprises capturing the parameter(s) of the patient into a predefined categories, and each category is assigned a score, i.e., the corresponding composite score (for example, the corresponding composite score for a category “good” may be 80, the corresponding composite score for a category “moderate” may be 50, and the corresponding composite score for a category “poor” may be 30). Now, continuing with the above example related to the first set of preset rules, where for assessing the Disease A, number of hours of physical activity, number of hours of meditation, and number of hours of sleep are relevant parameters, say for example, the corresponding composite score for ‘number of hours of physical activity’ is 50, the ‘number of hours of meditation’ is given 30, and the ‘number of hours of sleep’ is 80. Further, the second set of pre-set rules comprises multiplying each corresponding composite score with the respective weightage. Therefore, an intermediate output after multiplication of the corresponding composite scores with the respective weightages would be:
number of hours of physical activity: 50*50%=25
number of hours of meditation: 30*30%=9
number of hours of sleep: 80*20%=16

Further, the health wellness score of the user is the weighted average of all these intermediate outputs for all parameters, i.e., (25+9+16)/3=16.67

A person skilled in the art would appreciate that the above values are for understanding purposes only and do not restrict or limit the disclosure in any possible manner.

Also, in an event the processing unit is also configured to categorize the user into one or more health categories based at least on the health-wellness score of the user. In an exemplary implementation, each health category from the one or more health categories may be one of an ideal category, optimal category and sub-optimal category, but the disclosure is not limited thereto.

After automatically generating the health-wellness score of the user, the processing unit [104] is then configured to provide, to at least one of the user and a healthcare provider, the true health-wellness status of the user based on the health-wellness score of the user.

In an implementation, the true health-wellness status of the user may be provided based on the one or more health categories in which the user is categorized. For instance, based on the category the user fall into, one or more remedial measures may be suggested along with the true health-wellness status by taking into account the user's age, gender, lifestyle, comorbidities etc. and the user may be followed up for chronic care management. Also, in an implementation, the true health-wellness status is provided to at least one of the user and the healthcare provider using a colour coded scale with pointer to represent at least the ideal, optimal and sub-optimal health categories, or in any manner that it is easily understandable by the user. Further in an event, as and when the health category is updated, the colour coded scale may automatically represent the updated health category. The colour coded scale may also have a time stamp to keep track of when the last health category was updated. Also, in an implementation the true health-wellness status of the user may be provided at least as a progress bar with overall health-wellness score. Also, the true health-wellness status of the user is provided in one of a real time and a regular pre-defined interval. Also, in an implementation the true health-wellness status is provided via a representation indicating at least one of one or more health goal scores, and the health-wellness score of the user. The one or more health goal scores and the health-wellness score scores may be provided as percentages, visual scales, colour gradients, and/or charts etc. Also, each health goal score from the one or more health goal scores indicates a progress of the user with respect to one or more pre-defined health goals. The one or more health goal scores are determined based on the health-wellness score of the user and the one or more pre-defined health goals. Also, the one or more one or more pre-defined health goals are determined based on a manual input received from at least one of the user and the healthcare provider of the user.

Further, along with the true health-wellness status of the user, the processing unit [104] is also configured to provide one or more recommendations for at least one of one or more follow up consultations, one or more health investigations and one or more comorbidities based on the at least one of the one or more health goal scores and the health-wellness score of the user.

Also, in an implementation the processing unit [104] is further configured to provide, along with the true health-wellness status of the user, at least one of one or more suggested activities, one or more suggested precautions, one or more suggested treatment options, and one or more emergency alerts based on the at least one of the one or more health goals scores and the health-wellness score of the user.

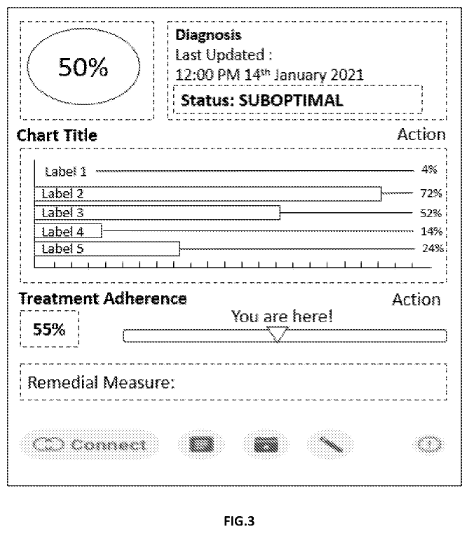

Also, the processing unit [104] in an implementation is configured to identify, one or more complications based at least on one of the one or more health goal scores and/or the health-wellness score of the user. Thereafter, in such implementation the processing unit [104] is configured to provide, the one or more emergency alerts for the one or more identified complications, along with the true health-wellness status of the user. Therefore, in an example, a representation may be displayed on the user device, to provide the true health-wellness status, along with one or more key health indicators of the user, medication adherence details, health goals etc., where the status may be displayed as scores, colour gradients, visual scales, charts etc. using pop-ups. Also, in one other example, the representation may be displayed on the user device, to provide one or more scenarios relevant to one or more treatment options provided to the user, to support decision making and treatment adherence at user's end. Further in one other example, the representation may be displayed on the user device, to provide apart from a primary disease/health condition, an information about user's/patient's comorbidities. In an implementation, the representation may be displayed on the user interface [106] of the system [100], and the same may be communicated further to the user device via the transceiver unit [102]. Furthermore, referring to FIG. 3, an exemplary representation providing an exemplary true health-wellness status is depicted in accordance with exemplary embodiments of the present invention.

In an implementation, the processing unit [104] is configured to receive from the storage unit [108], a content data, wherein the content data comprises at least one of a disease-related information and the one or more medical guidelines. This content data is a pre-configured information related to one or more known diseases or complications that may arise in the body of any person. For example, the content data may be in terms of one or more parameters related to body sugar levels for the purpose of treating diabetes. The information, thus, in this example, may suggest what is a safe range of body sugar level for a person, and what is an unsafe range of body sugar level for a person, so as to suggest output to a user who is assessing a person for diabetes. A person skilled in the art would appreciate that above example is provided for understanding purposes and does not limit or restrict this disclosure in any possible manner. Thus, the processing unit [104] is further configured to determine an assessment of the user for one or more diseases, based on the content data. And further, the processing unit [104] is configured to provide an output related to a disease probability, to at least one of the electronic device, and a healthcare provider device, based on the true health wellness status of the user and the assessment of the user. Further, based on the true health wellness status of the user, a treatment decision may be made by the user or the healthcare provider, etc. As shown in FIG. 4, the treatment decision [406] may comprise nutrition [422], physical activity [424], medical and surgical management [426], counselling [428], etc.

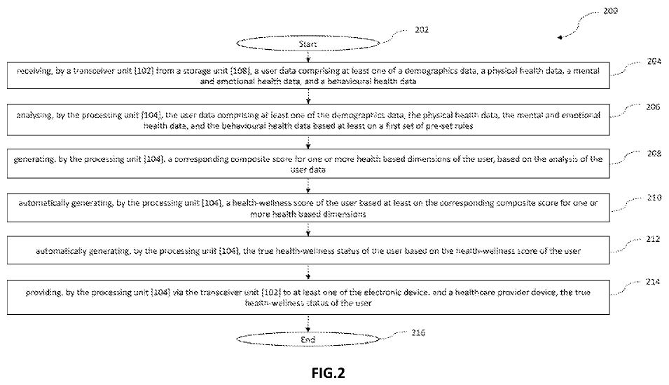

Referring to FIG. 2 an exemplary method flow diagram [200], for providing a true health-wellness status of a user, in accordance with exemplary embodiments of the present invention is shown. The present disclosure via the true health-wellness status provides two levels of information, i.e., 1) a holistic, general true health-wellness status/health-wellness score that considers multiple dimensions of health of an individual, and 2) a specific health and wellness score/status that could be pertaining to individual dimensions of health or the disease/condition that the individual has.

In an implementation the method is performed by the system [100]. Further, in an implementation, the system [100] may be present in a server device to implement the features of the present invention. Also, as shown in FIG. 2, the method starts at step [202], upon a diagnosis of a health condition and/or a disease (for e.g., diabetes but not limited thereto) and a selection of an option (such as a suitable care service) for implementation of the features of the present disclosure.

Thereafter at step [204] the method comprises receiving, by the transceiver unit [102] from the storage unit [106], a user data comprising at least one of a demographics data, a physical health data, a mental and emotional health data, and a behavioural health data. In an implementation, the demographics data, the physical health data, the mental and emotional health data, and the behavioural health data further comprises one or more parameters associated with the user. In an implementation, the user data is provided by the user via the electronic device. In an implementation, the user data is provided by a healthcare provider via the electronic device and/or via any other smart device connected with the electronic device. In an implementation the transceiver unit [102] receives the data at a wellness configurator unit. In an implementation, the wellness configurator unit may be a unit similar to the processing unit [104] and/or may be implemented in the processing unit [104]. The demographics data associated with the user includes but not limited to a data related to the user that is determined based on a study of a population-based on factors such as age, race, and gender etc. Also, the demographic data comprises at least a socio-economic status of the user, where in an example the socioeconomic status/information may include a data determined based on an employment, education, income, marriage rates, birth and death rates, and such other factors.

Also, in an implementation, the one or more parameters are configurable based on one of an automatic input and a manual input. For example, the user of the system [100] may select the parameter(s) to focus on while assessing the health of the assessee (i.e., the one whose health is assessed). Also, the user of the system [100] may be the same person whose health is assessed or may be a healthcare provider who is providing services to the user by using the system [100]. In an example, the user may select one or more parameters to focus on, such as Hemoglobin (Hb) level of the blood of the assessee, blood pressure of the assessee, oxygen level of the assessee, etc. This may be done via manual input of the user. Further, in another implementation, the user may select a disease to assess in the assessee. By simply selecting an option of that disease, the system [100] automatically selects corresponding one or more parameters to focus on.

Further, at step [206] the method comprises receiving, by the transceiver unit [102] from the storage unit [106], a physical health data associated with the user, wherein the physical health data comprises at least an anthropometry data, a general physical examination data and a systemic examination data etc. Next, at step [208] the method comprises receiving, by the transceiver unit [102] from the storage unit [106], a mental and emotional health data, and a behavioural health data associated with the user. The behavioural health data comprises at least a substance abuse data and a treatment adherence data.

Also, in an implementation, the user data is based on a primary diagnosis and other comorbidities of the user to make the assessment patient/user centric and customized for each user's/individual's health need. Further, the data received by the transceiver unit [102] from the storage unit [106] is used to determine a health-wellness score (for instance a general or a disease specific health-wellness score) of the user, and in an implementation the storage unit [106] may receive such data (i.e., the data to be transmitted to the transceiver unit [102]) from various sources such as including but not limited to one or more medical history forms provided by the user and/or the healthcare provider, one or more IoT and Point of Care devices, one or more validated assessment tools/questionnaires, one or more Lab reports, and one or more subjective inputs from Clinical Staff, one or more wearable gadgets of the user, etc. In an implementation, the storage unit [106] receives the data via the transceiver unit [102] and/or the processing unit [104]. Therefore, the data may be received at the transceiver unit [102] from various sources that may be device based sources and/or device-less sources. Also, the data received by the transceiver unit [102] from the storage unit [106] includes both subjective and objective data in order to obtain a result that is truly multidimensional. For example, the subjective and objective data may be obtained from clinical examination, medical records, lab investigations, IoT and Point of Care (POC) devices etc., where one or more members of a Care Team may coordinate with the user to schedule activities to obtain information from above mentioned sources.

Next, after receiving the data from the storage unit [106], the transceiver unit [102] provides the received user data to the processing unit [104] (say for e.g., to the Wellness Configurator Unit). Next, at step [210] the method comprises analysing, by the processing unit [104] (say for e.g., by the Wellness Configurator Unit), the received user data, i.e., at least one of the demographics data, the physical health data, the mental and emotional health data and/or the behavioural health data based at least on a first set of pre-set rules to generate a corresponding composite score for one or more health based dimensions of the user. The first set of pre-set rules comprises selecting the parameter(s) to assess the patient based on a disease condition of the patient. Further, the first set of pre-set rules comprises assigning a weightage to each of the selected parameter(s). These weightages may be assigned by a physician or a healthcare provider, or may be system generated based on historically assigned weightages. The corresponding composite score for one or more health based dimensions is generated to determine the health-wellness score of the user. In an implementation, the corresponding composite score for the one or more health based dimensions is based on at least one of a corresponding score-weightage for each parameter of the one or more parameters. The analysis may be based on at least one of the one or more medical guidelines and a medical history of the user. The medical history of the user may also be based on a family medical history of the user. For example, a user selects “Diabetes” as the disease for assessment. From the general medical guidelines such as a set of international guidelines, or general medical practices guidelines in a country, etc., two standard tests are prescribed, say Test A and Test B. However, from the family medical history of the user or the medical history of the user, it is suggested that Test C should also be performed for the assessment of diabetes in the user's specific case. Thus, it may be notified to the user by, for example, generating a prompt by the system that Test C is also suggested to be taken by the user. Thus, in an implementation, the user can select an option indicating whether the user wants to take the suggested test into consideration or continue with the analysis based on standard tests only. In another implementation, the user is not given an option to select the test consideration, but the prompt only contains an information informing the user that parameters related to the Test C are also taken into consideration while generating results.

Based on the analysis, the processing unit [104] assigns a weightage to the one or more parameters present in the received data to determine the corresponding composite score for one or more health based dimensions (i.e. to further determine the health-wellness score), wherein initially such weightage may be assigned for determining the corresponding composite score for one or more health based dimensions (or the health-wellness score) at a beginning level and the weightage may be then modified periodically and/or dynamically as per the user's health progress, so that the composite score for one or more health based dimensions (or the user's health-wellness score) may be determined and/or tracked effectively. Here, the corresponding composite score for the one or more health based dimensions is updated periodically based on a periodical update in the user data. In an implementation, the user data is updated periodically at regular or irregular intervals. In an implementation, the updating of the corresponding composite score for the one or more health based dimensions and the updating of the user data are synchronized in a manner that the corresponding composite score(s) is updated as and when the user data is updated. Also, in an implementation processing unit [104] (i.e., a wellness assessment unit) receives the weighted values of the one or more parameters present in the user data along with respective one or more baseline values of said one or more parameters and one or more target values for of said one or more parameters. The Wellness Assessment Unit may be a unit similar to the processing unit [104] and implements the features of the present invention. Further, in the given implementation the processing unit [104] (i.e., the Wellness Assessment Unit) may be configured to provide the composite score for one or more health based dimensions (or the health-wellness score) for the user based at least on an analysis of the weighted values of the one or more parameters present in the received data with respect to the one or more baseline values of said one or more parameters and the one or more target values for of said one or more parameters.

Also, in an implementation the received data i.e., the demographics data, the physical health data, the mental and emotional health data and/or the behavioural health data are analysed based on a patient health data of one or more patients similar to the user. For example, if the user is diagnosed with diabetes or a particular health condition, then the demographics data, the physical health data, the mental and emotional health data and/or the behavioural health data of such user are analysed based on a patient health data of one or more patients that at least are also diagnosed with diabetes or a particular health condition, and the first set of pre-set rules. More specifically, the processing unit [104] compiles and scores the demographics data, the physical health data, the mental and emotional health data and/or the behavioural health data based at least on the analysis of the demographics data, the physical health data, the mental and emotional health data and/or the behavioural health data, wherein the analysis is based on the first set of pre-set rules and the patient health data of the one or more patients that are also diagnosed with the same disease and/or same health condition as that of the user, to generate the composite score for the one or more health based dimensions of the user.

Further, after generating the corresponding composite score for the one or more health based dimensions of the user, at step [212] the method comprises automatically generating, by the processing unit [104], the health-wellness score of the user based on a weighting of the corresponding composite score, wherein the weighting of the composite score is based on a second set of pre-set rules. The second set of pre-set rules comprises capturing the parameter(s) of the patient into a predefined categories, and each category is assigned a score, i.e., the corresponding composite score. Further, the second set of pre-set rules comprises multiplying each corresponding composite score with the respective weightage. Further, an intermediate output after multiplication of the corresponding composite scores with the respective weightages is determined. Further, the health wellness score of the user is determined based on the weighted average of all the intermediate outputs for all parameters, as explained above in this disclosure.

Also, in an implementation, the method further comprises categorizing the user into one or more health categories based at least on the health-wellness score of the user. In an exemplary implementation, each health category from the one or more health categories may be one of an ideal category, optimal category and sub-optimal category, but the disclosure is not limited thereto.

Next, after automatically generating the health-wellness score of the user, at step [214] the method comprises providing, by the processing unit [104] via the transceiver unit [102] to at least one of the user and a healthcare provider, the true health-wellness status of the user based on the health-wellness score of the user.

In an implementation, the true health-wellness status of the user may be provided based on the one or more health categories in which the user is categorized. For instance, based on the category the user fall into, one or more remedial measures may be suggested along with the true health-wellness status by taking into account the user's age, gender, lifestyle, comorbidities etc. and the user may be followed up for chronic care management. Also, in an implementation the true health-wellness status is provided to at least one of the user and the healthcare provider using a colour coded scale with pointer to represent at least the ideal, optimal and sub-optimal health categories, or in any manner that it is easily understandable by the user. Further in an event, as and when the health category is updated, the colour coded scale may automatically represent the updated health category. The colour coded scale may also have a time stamp to keep track of when the last health category was updated. Also, in an implementation the true health-wellness status of the user may be provided at least as a progress bar with overall health-wellness score. Also, the true health-wellness status of the user is provided in one of a real time and a regular pre-defined interval. Also, in an implementation the true health-wellness status is provided via a representation indicating at least one of one or more health goal scores, and the health-wellness score of the user. The one or more health goal scores and the health-wellness score scores may be provided as percentages, visual scales, colour gradients, and/or charts etc. Also, each health goal score from the one or more health goal scores indicates a progress of the user with respect to one or more pre-defined health goals. The one or more health goal scores are determined based on the health-wellness score of the user and the one or more pre-defined health goals. Also, the one or more one or more pre-defined health goals are determined based on a manual input received from at least one of the user and the healthcare provider of the user.

Also, the process of providing at least one of the one or more health goal scores and the health-wellness score of the user further comprises providing one or more recommendations for at least one of one or more follow up consultations, one or more health investigations and one or more comorbidities based on the at least one of the one or more health goals scores and the health-wellness score of the user. Also, in one other implementation the process of providing at least one of the one or more health goal scores and the health-wellness score of the user further comprises providing at least one of one or more suggested activities, one or more suggested precautions, one or more suggested treatment options, and one or more emergency alerts based on the at least one of the one or more health goal scores and the health-wellness score of the user.

Further, in an implementation, the method also encompasses identifying, by the processing unit [104], one or more complications based at least on one of the one or more health goal scores and/or the health-wellness score of the user. In the given implementation, the method then leads to providing, by the processing unit [104], the one or more emergency alerts for the one or more identified complications, along with the true health-wellness status of the user.

In an implementation, the method comprises receiving, by the processing unit [104] from the storage unit [108], a content data, wherein the content data comprises at least one of a disease-related information and one or more medical guidelines. This content data is a pre-configured information related to one or more known diseases or complications that may arise in the body of any person. For example, the content data may be in terms of one or more parameters related to body sugar levels for the purpose of treating diabetes. The information, thus, in this example, may suggest what is a safe range of body sugar level for a person, and what is an unsafe range of body sugar level for a person, so as to suggest output to a user who is assessing a person for diabetes. A person skilled in the art would appreciate that above example is provided for understanding purposes and does not limit or restrict this disclosure in any possible manner. Thus, the processing unit [104] further determines an assessment of the user for one or more diseases, based on the content data. And further, the processing unit [104] provides an output related to a disease probability, to at least one of the electronic device, and a healthcare provider device, based on the true health wellness status of the user and the assessment of the user.

After providing the true health-wellness status of the user, the method terminates at step [216].

### Use Cases

There are a number of use cases of the solution as depicted in the present disclosure, but the exemplary steps that may be required to provide one of an exemplary use case of providing a true health-wellness status of a user are as follows:
1. In order to ensure patient privacy and security of data, there is a need for the user to log in using their unique credentials to a digital platform that is configured to implement the features of the present invention. Therefore, at step 1, the user logs in to the digital platform accessed on a user device, by entering his/her username which may be, but not limited to, a platform-specific username, or a phone number, or an email address, etc. The system [100] while logging in asks for a password which could be created by the user, or the system [100] sends an OTP to the phone number/email address and the user can login using that. The creation of the password may be a one-time process, and once created, the same username and password may be used by the user to log in to the digital platform on subsequent times.
2. Thereafter at step 2, a user registration process may be initiated, where the user may register himself by filling out basic information like name, gender, age, height, weight etc. These basic user details help the system [100] to assign the user to specific populations for the purpose of calculating risks, normal laboratory ranges etc. Also, age or gender specific evaluations may also be suggested based on these basic details.
   These basic details of the user may be used in various cases, for example, for configuring the parameters automatically for assessing the user or for providing weightage to the parameters. In an exemplary case, say, a paralytic person is going for physiotherapy. Also, say, the age of that person is 70 years. In that case, the parameters for wellness score may be different from those of a healthy person. In such a case, say, even if the paralytic person is able to move his hands by 5 cm, may be considered healthy, and may not be expected to run or jump like some other people. Based on such user details, the health goals, suggested activities for the user may also vary. This is how the customisation of health-wellness status may be performed by the system [100].
3. Further, at step 3, a data input for detailed health/disease status information of user is provided to the system [100]. More specifically, detailed information on general health status or on a disease status (for e.g., diabetes status) may require data from multiple sources including both subjective and objective data sources. These data may include data related to lifestyle, daily routine, an information obtained by way of questionnaires and/or medical history such as clinical examinations, lab investigations, past records etc. Therefore, the user and/or one or more healthcare workers may provide to the system [100], one or more past medical records like prescriptions, lab reports, clinical examination based data etc., where the provided records are then validated and converted to structured data. Also, in an event, a health related information captured via IoT and/or POC devices may be also be received at the system [100]. The POC devices are efficient source of objective health related data and may provide real time data about health parameters for tracking patient progress. In an example, the user/patient may be suggested/prescribed POC devices and the information captured by such suggested/prescribed POC devices is then provided to the system [100] for further processing. Also, an objective information from synced POC devices may be automatically shared with the system [100].
4. Next, at step 4, by the implementing the features of the present disclosure a detailed true health-wellness status information may be determined based on the received data, and the detailed true health-wellness status information is then provided to the user and/or a health care provider. More specifically, based on the implementation of the features of the present disclosure, the received data is compiled and scored by the system [100] according to pre-set rules to create composite scores for one or more health based dimensions. Further these composite scores are weighted by the system [100] based on one or more second pre-set rules to create a single true health-wellness status. In an implementation, the determined true health-wellness status may be accompanied by a suitable explanation, one or more remedial measures and/or one or more suggested actions to make the system [100] truly actionable. Also, in an implementation the true health-wellness status of the user may be provided based on one or more health categories in which the user is categorized based on a health-wellness score of the user, where each health category from the one or more health categories may be one of an ideal category, optimal category and sub-optimal category. In an event if a user's health category is ideal, the user may be shown a message to repeat the process to generate the true health-wellness status after a certain fixed time interval. Also, the user may be suggested to undergo a preventive health check-up depending on their age, gender, lifestyle, family history (of hypertension, diabetes, stroke, heart attack) etc. The user may also be given an option to connect to a healthcare team if the user need further information and the healthcare team may be notified that the user needs to repeat healthcare assessment after a certain fixed time interval. In an event the system [100] may store this data to use it for comparison during the generation of next true health-wellness status.
   Further, in an event if a user's health category is optimal, the user may be provided with a brief breakup of their scores and suggestions to improve the same. Also, the user may be given an option to connect to the healthcare team via call/SMS/in-app messages etc. if the user needs further information, and the healthcare team may be notified that the user is in optimal zone and needs to repeat healthcare assessment after a certain fixed time interval. Also, in case the user connects to the healthcare team, the user would be briefed about the further course of action suggested and the healthcare coach may suggest basic interventions to the user. Also, in an event the healthcare team member may also answer any question that the user has and onboard the user for further evaluation if the user wants to do so. Further in an event system [100] may store this data to use it for comparison during the generation of next true health-wellness status.
   Also, in an event if a user's health category is sub-optimal, the user may be provided with a brief breakup of their scores and suggestions to improve the same. In an event, the healthcare team may also be notified that the user is in sub-optimal zone and the user may be then contacted by a healthcare team member and briefed about the further course of action suggested. Also, in an event the healthcare team member may also answer any question that the user has and onboard the user for further evaluation if the user wants to do so. Further in an event system [100] may store this data to use it for comparison during the generation of next true health-wellness status.
5. Also, at step 5, along with the true health-wellness status, the user may be able to view an option to learn more about their health condition, existing guidelines, treatment options etc. Therefore, the user is equipped with knowledge for self-management, decision support with respect to his treatment. In an implementation the generated true health-wellness status may be displayed on a circular progress bar with a pop-up window containing the details. The status may comprise of patient's key indicators, medication indicators, health goals etc. Also, the pop-up window may have information in the form of bar charts, line trends, visual scales etc. Therefore, if the user wish to know about his health in much more detail, he is able to do so in a manner that is easy to understand, track and act upon. Most individuals with chronic conditions/diseases like diabetes etc. may like to know their disease status-if disease is controlled or not, whether they are at risk of any complications etc. The true health-wellness status also helps them to track their progress with respect to their health goals.
6. Also, at step 6, the system [100] may provide care plan suggestion(s) based on the categories like: a variant of the disease (clinical onset, grades from pathological reports etc.), a stage and/or severity of disease at which the user/patient currently is, and patient profiling with help of anthropometry details and age, gender, confounding factors etc.
7. Next, at step 7, using data mining and AI technologies, the system [100] may display one or more predictions of health outcomes based on whether or not an intervention is adopted. Therefore, the user may benefit from understanding how a given intervention may impact the user's health status. Showing the users, a comparison between his before and after-intervention states may help him understand the impact better.
8. Also, at step 11, short, mid-and long-term health goals may be decided jointly by the user/patient and the care team considering user's/patient's disease, severity of disease, confounding factors like lifestyle etc. Further, the user and/or the healthcare workers may then view along with the health status, the user's progress with respect to achieving his health goals. In an event the progress may be represented as percentages, visual scales, colour gradients, charts etc.
9. Next at step 12, system [100] may also generate alerts and provide the generated alerts at least to healthcare provider/healthcare coach as critical value triggers sent to healthcare workers in real time can ensure timely intervention. More specifically, if during any periodic assessment, a user logs in a critical value (critical value for different health parameters may be predetermined and fed into the system [100]), the system [100] automatically sends alerts to specific healthcare team and the specific healthcare team member may then connect to the user and guide them on further needful action. Also, in an event the system [100] may also generate and provide one or more notifications for performing periodic health assessment based on the health-wellness score of the user. Also in an instance, depending on a predetermined periodicity, the system [100] may display one or more notifications to the user for scheduling appointments.

Thus, the system for providing a true health-wellness status of a user involves a 3 stage approach, where, the first stage is the “assessment stage” at which the user is assessed to understand the health needs and goals for the user. The second stage is the “configuration stage” at which relevant care indicators or the parameters are identified and/or selected for assessing the user. The third stage is the “visualization stage” at which the user is provided the output of the assessment of the assessee, i.e., the health-wellness status of the assessee, along with the health goals, suggested activities, consultation frequencies, indication of complications, etc.

Thus, the present invention provides a novel solution of providing a true health-wellness status of a user. The true health-wellness status as provided by the present invention includes all aspects of health providing a holistic, multidimensional view of health including efficient and rapid management of the condition, improving patient experience etc. Also, the present invention uses simple examinations and easily available tests for initial assessment. It uses advanced tests only in cases where such tests are absolutely necessary and scientifically indicated. This makes the present invention much more accessible to a wide variety of population. Also, the present invention is hybrid i.e. it takes into account both subjective and objective data. It uses data from both devices (IoT's, PoC) as well as device-less sources like questionnaires etc. to calculate a holistic score, thereby reducing dependence on device and ensuring that:
1. The subjective experiences are taken into account
2. The exercise of determining the true health-wellness status can be repeated periodically with ease
3. Cost of determining the disease specific health-wellness is significantly reduced

Although the present invention is quite useful for healthcare providers to monitor patient progress, evaluating and comparing treatments, identifying efficiencies, assessing risks, and implementing proven solutions on a continuous basis, the core objective of the present invention is to cater to individuals/patients i.e. inform them about their health status and motivate them to adopt practices beneficial for their health in achieving the set health goals. Furthermore, the present invention provides personalized wellness experience to the users, as every individual has a unique health need and very different set of external factors that affect their health. The present invention takes into account these differences while providing information related to their health. Also, the present solution provides a single, holistic score and various specific scores like physical health score, mental health score, disease-specific score etc. Furthermore, the present invention displays health score in a simple, easy to understand, interactive, and actionable representation of health data. The present invention also presents health status along with real time support from a healthcare team and patient education materials. Additionally, the present invention provides a predictive risk assessment for comorbidities. Therefore, the present invention overcomes the limitations of the existing solutions and has a number of technical advancements over the currently known solutions.

While considerable emphasis has been placed herein on the preferred embodiments, it will be appreciated that many embodiments can be made and that many changes can be made in the preferred embodiments without departing from the principles of the invention. These and other changes in the preferred embodiments of the invention will be apparent to those skilled in the art from the disclosure herein, whereby it is to be distinctly understood that the foregoing descriptive matter to be implemented merely as illustrative of the invention and not as limitation.

We claim:

1. A method for providing a true health-wellness status of a user of an electronic device, the method comprising:
receiving, by a transceiver unit [102] from a storage unit [108], a user data comprising at least one of a demographics data, a physical health data, a mental and emotional health data, and a behavioural health data;
analysing, by the processing unit [104], the user data comprising at least one of the demographics data, the physical health data, the mental and emotional health data, and the behavioural health data, based at least on a first set of pre-set rules;
generating, by the processing unit [104], a corresponding composite score for one or more health based dimensions of the user, based on the analysis of the user data;
automatically generating, by the processing unit [104], a health-wellness score of the user based at least on the corresponding composite score for one or more health based dimensions;
automatically generating, by the processing unit [104], the true health-wellness status of the user based on the health-wellness score of the user; and
providing, by the processing unit [104] via the transceiver unit [102] to at least one of the electronic device, and a healthcare provider device, the true health-wellness status of the user.

2. The method as claimed in claim 1, wherein the at least one of the demographics data, the physical health data, the mental and emotional health data, and the behavioural health data further comprises one or more parameters associated with the user.

3. The method as claimed in claim 2, wherein the corresponding composite score for the one or more health based dimensions is based on at least one of a corresponding score-weightage for each parameter of the one or more parameters.

4. The method as claimed in claim 2, wherein the one or more parameters are configurable based on one of an automatic input and a manual input.

5. The method as claimed in claim 1, wherein the physical health data comprises at least one of an anthropometry data, a general physical examination data, and a systemic examination data.

6. The method as claimed in claim 1, wherein the behavioural health data comprises at least one of a substance abuse data and a treatment adherence data.

7. The method as claimed in claim 1, wherein the user data is based on a primary diagnosis of the user and diagnosis of one or more comorbidities of the user.

8. The method as claimed in claim 1, wherein the user data is provided by the user via the electronic device.

9. The method as claimed in claim 1, wherein the automatically generating, by the processing unit [104], the health-wellness score of the user based at least on the corresponding composite score further comprises:
automatically generating, by the processing unit [104], the health-wellness score of the user based on a weighting of the corresponding composite score for one or more health based dimensions using a second set of pre-set rules.

10. The method as claimed in claim 3, wherein the corresponding composite score for the one or more health based dimensions is updated periodically based on a periodical update in the user data.

11. The method as claimed in claim 10, wherein the generating, by the processing unit [104], the corresponding composite score for the one or more health based dimensions of the user, is further based on at least one of a baseline value of said each parameter of the one or more parameters and a target value of said each parameter of the one or more parameters.

12. The method as claimed in claim 1, wherein the analysis of the user data is further based on a patient health data of one or more patients similar to the user.

13. The method as claimed in claim 1, wherein the true health-wellness status is provided via a representation indicating at least one of one or more health goal scores, and the health-wellness score of the user.

14. The method as claimed in claim 13, the method further comprising:
providing, by the transceiver unit [102] to at least one of the electronic device, and a healthcare provider device, one or more recommendations for at least one of one or more follow up consultations, one or more health investigations and one or more comorbidities based on at least one of the one or more health goal scores and the health-wellness score of the user.

15. The method as claimed in claim 13, the method further comprising:
providing, by the transceiver unit [102] to at least one of the electronic device, and the healthcare provider device, at least one of one or more suggested activities, one or more suggested precautions, one or more suggested treatment options, and one or more emergency alerts based on at least one of the one or more health goal scores and the health-wellness score of the user.

16. The method as claimed in claim 15, the method further comprising:
identifying, by the processing unit [104], one or more complications based on at least one of the one or more health goal scores and the health-wellness score of the user; and
providing, by the processing unit [104] via the transceiver unit [102] to at least one of the electronic device, and a healthcare provider device, the one or more emergency alerts for the one or more identified complications.

17. The method as claimed in claim 1, the method comprising:
receiving, at the processing unit [104] from the storage unit [108], a content data, wherein the content data comprises at least one of a disease-related information and one or more medical guidelines;
determining, by the processing unit [104], an assessment of the user for one or more diseases, based on the content data; and
providing, via the transceiver unit [102], an output related to a disease probability, to at least one of the electronic device, and a healthcare provider device, based on the true health wellness status of the user and the assessment of the user.

18. The method as claimed in claim 17, wherein the analysing, by the processing unit [104], the user data, is based on at least one of the one or more medical guidelines and a medical history of the user.

19. A system for providing a true health-wellness status of a user of an electronic device, the system comprising:
a transceiver unit [102];
a storage unit [108]; and
a processing unit [104] connected to the transceiver unit [102];
wherein the transceiver unit [102] is configured to:
receive, from the storage unit [108], a user data comprising at least one of a demographics data, a physical health data, a mental and emotional health data, and a behavioural health data;
the processing unit [104] is configured to:
analyse the user data comprising at least one of the demographics data, the physical health data, the mental and emotional health data, and the behavioural health data, based at least on a first set of pre-set rules;
generate a corresponding composite score for one or more health based dimensions of the user, based on the analysis of the user data;
automatically generate a health-wellness score of the user based at least on the corresponding composite score for one or more health based dimensions;
automatically generate the true health-wellness status of the user based on the health-wellness score of the user; and
provide, via the transceiver unit [102], to at least one of the electronic device, and a healthcare provider device, the true health-wellness status of the user.

20. The system as claimed in claim 19, wherein the at least one of the demographics data, the physical health data, the mental and emotional health data, and the behavioural health data further comprises one or more parameters associated with the user.

21. The system as claimed in claim 20, wherein the corresponding composite score for the one or more health based dimensions is based on at least one of a corresponding score-weightage for each parameter of the one or more parameters.

22. The system as claimed in claim 20, wherein the one or more parameters are configurable based on one of an automatic input and a manual input.

23. The system as claimed in claim 19, wherein the physical health data comprises at least one of an anthropometry data, a general physical examination data and a systemic examination data.

24. The system as claimed in claim 19, wherein the behavioural health data comprises at least one of a substance abuse data and a treatment adherence data.

25. The system as claimed in claim 19, wherein the user data is based on a primary diagnosis of the user and diagnosis of one or more comorbidities of the user.

26. The system as claimed in claim 19, wherein the user data is provided by the user via the electronic device.

27. The system as claimed in claim 19, wherein the processing unit [104], to automatically generate the health-wellness score of the user based at least on the corresponding composite score, is further configured to:
automatically generate the health-wellness score of the user based on a weighting of the corresponding composite score for one or more health based dimensions using a second set of pre-set rules.

28. The system as claimed in claim 21, wherein the corresponding composite score for the one or more health based dimensions is updated periodically based on a periodical update in the user data.

29. The system as claimed in claim 28, wherein the processing unit [104], for generating the corresponding composite score for the one or more health based dimensions of the user, uses at least one of a baseline value of said each parameter of the one or more parameters, and a target value of said each parameter of the one or more parameters.

30. The system as claimed in claim 19, wherein the analysis of the user data is further based on a patient health data of one or more patients similar to the user.

31. The system as claimed in claim 19, wherein the true health-wellness status is provided via a representation indicating at least one of one or more health goal scores, and the health-wellness score of the user.

32. The system as claimed in claim 31, wherein the transceiver unit [102] is further configured to:
provide, to at least one of the electronic device, and a healthcare provider device, one or more recommendations for at least one of one or more follow up consultations, one or more health investigations and one or more comorbidities based on at least one of the one or more health goal scores and the health-wellness score of the user.

33. The system as claimed in claim 31, wherein the transceiver unit [102] is further configured to:
provide, to at least one of the electronic device, and the healthcare provider device, at least one of one or more suggested activities, one or more suggested precautions, one or more suggested treatment options, and one or more emergency alerts based on at least one of the one or more health goal scores and the health-wellness score of the user.

34. The system as claimed in claim 33, wherein the processing unit [104] is further configured to:
identify one or more complications based on at least one of the one or more health goal scores and the health-wellness score of the user; and
provide via the transceiver unit [102] to at least one of the electronic device, and a healthcare provider device, the one or more emergency alerts for the one or more identified complications.

35. The system as claimed in claim 19, wherein:
the processing unit [104] is configured to:
receive, from a storage unit [108], a content data, wherein the content data comprises at least one of a disease-related information and one or more medical guidelines;
determine an assessment of the user for one or more diseases, based on the content data; and
the transceiver unit [102] is configured to:
provide an output related to a disease probability, to at least one of the electronic device, and a healthcare provider device, based on the true health wellness status of the user and the assessment of the user.

36. The system as claimed in claim 35, wherein the analysing, by the processing unit [104], the user data, is based on at least one of the one or more medical guidelines and a medical history of the user.
